(19) United States
(12) Patent Application Publication
(10) Pub. No.: US 2020/0022592 A1
(43) Pub. Date: Jan. 23, 2020

(54) BLOOD PRESSURE MEASUREMENT TECHNIQUES AND DEVICES

(71) Applicant: Mayo Foundation for Medical Education and Research, Rochester, MN (US)

(72) Inventors: Pavol Sajgalik, Rochester, MN (US); Vaclav Kremen, Rochester, MN (US); Lyle D. Joyce, Rochester, MN (US); John A. Schirger, Rochester, MN (US); Bruce D. Johnson, Rochester, MN (US); Troy J. Cross, Rochester, MN (US); Vratislav Fabian, Prague (CZ)

(73) Assignee: Mayo Foundation for Medical Education and Research, Rochester, MN (US)

(21) Appl. No.: 16/477,408

(22) PCT Filed: Jan. 9, 2018
(86) PCT No.: PCT/US18/12898

§ 371 Date: Jul. 11, 2019

Related U.S. Application Data
(60) Provisional application No. 62/445,086, filed on Jan. 11, 2017.

(51) Int. Cl. A61B 5/0235; A61B 5/022

(52) U.S. Cl. CPC A61B 5/0235; A61B 5/0215; A61B 5/02225

(57) ABSTRACT

Non-invasive systems and techniques can be used to accurately measure blood pressures of patients being supported by continuous flow left ventricular assist devices. For example, this document describes cuff occlusion devices and methods for their use so that the blood pressure and pulsatility of patients with significantly reduced pulsatility as a result of continuous flow left ventricular assist device support can be accurately measured in a non-invasive fashion.

### CROSS REFERENCE TO RELATED APPLICATIONS

This application claims the benefit of U.S. Provisional Application No. 62/445,06, filed Jan. 11, 2017. The disclosure of the prior application is considered part of and is incorporated by reference in the disclosure of this application.

### BACKGROUND

### 1. Technical Field

This document relates to systems and methods for non-invasively measuring blood pressure. For example, this document relates to cuff occlusion devices and methods for their use so that the blood pressure of patients being supported by continuous flow left ventricular assist devices can be accurately measured in a non-invasive fashion.

### 2. Background Information

An estimated 5.8 million adults in the United States are currently living with heart failure (HF) and its prevalence is projected to increase to 25% by 2030. There is a broad clinical spectrum of HF, ranging from asymptomatic left ventricular dysfunction, symptomatic congestive HF, to end stage HF. A significant number of patients with congestive HF progress to require consideration of either cardiac transplantation or left ventricular assist device (LVAD) support.

A growing number of heart transplant candidates require long term implantable mechanical support, predominantly by LVAD, while they await cardiac transplantation, in so called bridge to transplant indications or for lifelong support as a destination therapy. The proportion of patients utilizing durable mechanical cardiac support increased from 14.7% in 2006-2007 to 41% in 2011-2013 in the USA. Data collected from 158 hospitals to the US national registry INTERMACS shows tremendous increase of LVAD implantations from 866 implants in 2009 to 2420 implants in 2013. With a survival rate of about 80% in the first year and 70% in the second year, LVAD therapy has evolved into a standard therapy for patients with advanced HF in last years.

### SUMMARY

Standard methods of non-invasive blood pressure (BP) measurement are not sufficiently reliable for BP measurement of patients supported by a continuous flow (CF) LVAD. That is at least in part the case because patients with a CF LVAD have significantly reduced pulsatility. Standard ausculatory methods or broadly used automatic oscillometric BP devices are generally not satisfactorily successful in the absence of pulsatile arterial flow.

This document describes systems and methods for non-invasively measuring BP. For example, this document describes cuff occlusion devices and methods for their use so that the BP of patients being supported by CF LVADs can be accurately measured in a non-invasive fashion.

In one aspect, this disclosure is directed to a system for measuring a blood pressure of a patient. Such a system includes: an inflatable cuff configured to releasably surround an arm of the patient and to occlude a brachial artery of the patient while the inflatable cuff is inflated; a pressure-regulating valve in fluid communication with the inflatable cuff; a second valve in fluid communication with the inflatable cuff and with the pressure-regulating valve; and a differential pressure sensor arranged to detect a blood pressure pulse wave measurement curve of the patient.

In another aspect, this disclosure is directed to a system for measuring a blood pressure of a patient. Such a system includes: an inflatable cuff configured to releasably surround an arm of the patient and to occlude a brachial artery of the patient while the inflatable cuff is inflated; a pressure-regulating valve through which air passes to inflate and deflate the inflatable cuff; a second valve in fluid communication with the inflatable cuff and with the pressure-regulating valve; and a differential pressure sensor arranged to detect a differential pressure of fluid lines on opposing sides of the second valve.

Either of the above systems for measuring a blood pressure of a patient may optionally include one or more of the following features. The system may also include a pump to supply air to inflate the inflatable cuff. The pressure-regulating valve may be located along an air supply line between the pump and the inflatable cuff. The system may also include a controller configured to receive signals from the differential pressure sensor and to determine the blood pressure based on the signals. In some embodiments, the controller controls opening and closing of the second valve. In particular embodiments, the controller controls the pressure-regulating valve to control air pressure in the inflatable cuff. In certain embodiments, the controller controls a pump that supplies air to inflate the inflatable cuff.

In another aspect, this disclosure is directed to a method of measuring blood pressure of a patient. The method includes: (i) inflating an inflatable cuff of a blood pressure measurement system to occlude a brachial artery of the patient; (ii) detecting, by a differential pressure sensor and while the brachial artery is at least partially occluded, a plurality of blood pressure pulse wave measurement curves; and (iii) determining the blood pressure based on the plurality of blood pressure pulse wave measurement curves. The blood pressure measurement system also includes: a pressure-regulating valve in fluid communication with the inflatable cuff; a second valve in fluid communication with the inflatable cuff and with the pressure-regulating valve; and the differential pressure sensor arranged to detect a blood pressure pulse wave measurement curve of the patient.

Such a method of measuring the blood pressure of a patient may optionally include one or more of the following features. The blood pressure measurement system may also include a controller that receives signals from the differential pressure sensor corresponding to the plurality of blood pressure pulse wave measurement curves, and the determining the blood pressure may be performed by the controller. The controller may control the pressure-regulating valve to vary air pressure in the inflatable cuff. The controller may control opening and closing of the second valve. The detecting the plurality of blood pressure pulse wave measurement curves may be performed while the second valve is closed and such that the differential pressure sensor measures a difference in pressures at opposing sides of the second valve. The detecting the plurality of blood pressure pulse wave measurement curves may be performed without pressure effects of air pressures that inflate the inflatable cuff.

Particular embodiments of the subject matter described in this document can be implemented to realize one or more of the following advantages. First, the devices and techniques described herein provide a higher level of BP measurement accuracy as compared to current non-invasive techniques for measuring BP of patient's supported by CF LVADs. Accordingly, better BP control can be achieved and the risk of adverse patient health events can be potentially mitigated. Second, the devices and techniques described herein are less complex and easier to use than current non-invasive techniques for measuring BP of patient's supported by CF LVADs. Therefore, the devices are advantageously potentially suitable for home use by a patient, leading to more frequent monitoring and better BP control. Moreover, in some embodiments provided herein BP can be accurately estimated in a minimally invasive fashion using the devices and methods. Such minimally invasive techniques can tend to reduce patient discomfort, recovery times and risks, and treatment costs.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims. While this specification contains many specific implementation details, these should not be construed as limitations on the scope of any invention or of what may be claimed, but rather as descriptions of features that may be specific to particular embodiments of particular inventions.

### DESCRIPTION OF THE DRAWINGS

FIG. 1 is a schematic illustration of an example blood pressure measurement system being used to measure the blood pressure of a patient in a non-invasive fashion.

FIG. 2 is a block diagram of an example controller of the blood pressure measurement system of FIG. 1, in accordance with some embodiments.

FIG. 3 shows an experimental setup for comparing the example controller of the blood pressure measurement system of FIG. 1 with an invasive intra-arterial blood pressure measurement technique.

FIGS. 4-8 shows data collected from experiments that were performed to prove the feasibility and to confirm the accuracy of the blood pressure measurement system of FIG. 1.

FIG. 9 illustrates the frequency characteristics of a blood pressure signal obtained invasively using an intra-arterial (I-A) sensor and characterized via a normalized power spectral density.

FIG. 10 illustrates the frequency characteristics of a blood pressure signal obtained non-invasively using novel measurement systems and techniques described herein and characterized via a normalized power spectral density.

FIGS. 11 and 12 illustrate the correlation between BP measurement results of: (i) invasively obtained I-A BP and (ii) non-invasively obtained BP (using the novel BP measurement systems described herein and a frequency domain spectral analysis approach).

Like reference numbers represent corresponding parts throughout.

### DETAILED DESCRIPTION

This document describes systems and methods for non-invasively measuring blood pressure (BP). For example, this document describes cuff occlusion devices and methods for their use so that the BP of patients being supported by CF LVADs can be accurately measured in a non-invasive fashion.

Standard methods of non-invasive blood BP measurement are not sufficiently reliable for patients supported by a CF LVAD. That is at least in part the case because patients with a CF LVAD have significantly reduced pulsatility. Standard ausculatory methods or broadly used automatic oscillometric BP devices are generally not satisfactorily successful in the absence of pulsatile arterial flow.

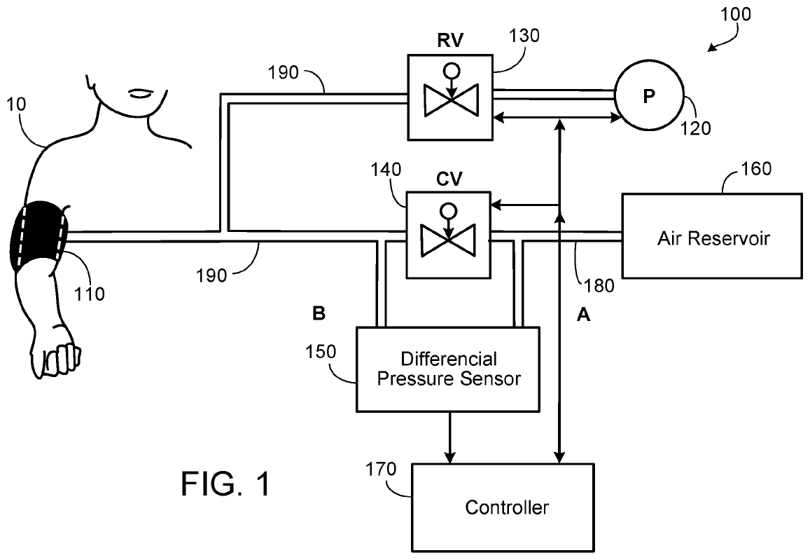

Referring to FIG. 1, a BP measurement system 100 can be used to accurately measure the BP of a patient 10. Moreover, even in the event patient 10 has a significantly reduced pulsatility (e.g., because patient 10 is supported by a CF LVAD), BP measurement system 100 can nevertheless accurately measure the BP of patient 10 in a non-invasive fashion.

Blood pressure measurement system 100 includes an inflatable cuff 110, a pump 120, a pressure-regulating valve 130, a closing valve 140, a differential pressure sensor 150, an air reservoir 160, a controller 170, a static pressure line 180, and an active pressure line 190.

Like a typical cuff of a conventional blood pressure measurement system, inflatable cuff 110 is configured to releasably surround an arm of patient 10 and to adjustably occlude a brachial artery of patient 10 while inflatable cuff 110 is inflated at varying levels of air pressure.

Pump 120 pressurizes the air supplied to inflatable cuff 110. In some embodiments, pump 120 is a manual pump (e.g., a bulb). In some embodiments, pump 120 is an electrically operated (e.g., battery operated or A/C operated) pump. In some such embodiments, the operations of pump 120 are automatically controlled by controller 170 in accordance with software and/or in accordance with user inputs provided via a user interface (UI) in communication with controller 170.

Pressure-regulating valve 130 is in fluid communication with pump 120 and with inflatable cuff 110 via active pressure line 190. Pressure-regulating valve 130 regulates the pressure of the air in inflatable cuff 110, and thereby regulates the extent to which the brachial artery of patient 10 is occluded. In some embodiments, pressure-regulating valve 130 is servo-operated and automatically controlled by controller 170 in accordance with software and/or in accordance with user inputs provided via a UI in communication with controller 170. Accordingly, controller 170 can be programmed and operated to controllably gradually reduce the inflation pressure of cuff 110 in a manner like or similar to the usual process for measuring BP using an automatic oscillometric BP device.

Closing valve 140 (also referred to herein as a “second valve”) is in fluid communication with inflatable cuff 110 and with pressure-regulating valve 130 via active pressure line 190. In some embodiments, closing valve 140 is a solenoid valve that is controlled by controller 170 to automatically operate the opening and closing of closing valve 140. Static pressure line 180 is connected to closing valve 140 on an opposing side of closing valve 140 in relation to active pressure line 190. That is, static pressure line 180 and active pressure line 190 are connected on opposing sides of closing valve 140. Accordingly, while closing valve 140 is closed, a pressure differential can exist between static pressure line 180 and active pressure line 190. Conversely, while closing valve 140 is open, static pressure line 180 and active pressure line 190 are in fluid communication via closing valve 140 such that the pressures in static pressure line 180 and active pressure line 190 are equalized while closing valve 140 is open.

In some embodiments, an optional air reservoir 160 is included. Air reservoir 160 can be any type of air containment device, i.e., a tube, a tank, a tubing network, and the like. Air reservoir 160 dead-ends static pressure line 180.

Differential pressure sensor 150 is arranged to measure a differential pressure of fluid lines on opposing sides of second valve 140. In the depicted embodiment, differential pressure sensor 150 is arranged to detect pressure differentials between static pressure line 180 and active pressure line 190. Accordingly, as described further below, differential pressure sensor 150 is arranged to measure a blood pressure pulse wave measurement curve of patient 10.

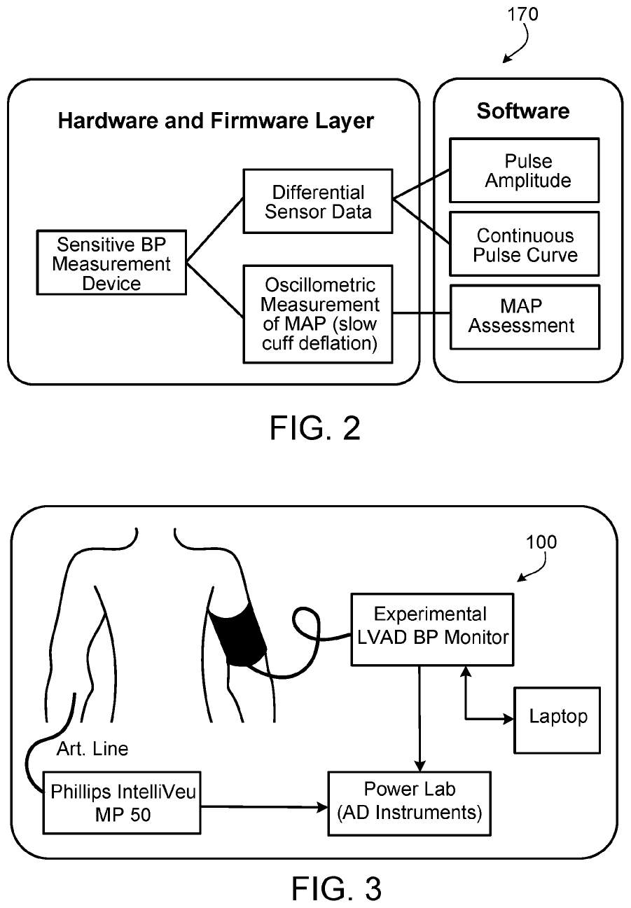

Also referring to FIG. 2, BP measurement system 100 includes controller 170. Controller 170 can include hardware (e.g., one or more processors, memory, UI components, power source(s), circuitry, etc.), firmware, and software. In the depicted embodiment, controller 170 is in electrical communication with pump 120, pressure-regulating valve 130, closing valve 140, and differential pressure sensor 150. Accordingly, controller 170 can control the operations of pump 120, pressure-regulating valve 130, closing valve 140, and differential pressure sensor 150. In some embodiments, controller 170 controls such devices in accordance with a set of executable instructions stored in memory that are executable by one or more processors of controller 170. In that fashion, controller 170 can automatically or semi-automatically operate BP measurement system 100. Moreover, controller 170 can be programmed to interpret input signals, e.g., from differential pressure sensor 150, so as to determine the BP of patient 10 based on a plurality of blood pressure pulse wave measurement curves detected by differential pressure sensor 150.

One reason that BP measurement system 100 can accurately measure the BP of patient 10 (even when patient 10 has a significantly reduced pulsatility) is because BP measurement system 100 uses a differential pressure measurement by which BP measurement system 100 detects the pulse amplitude (BP pulse wave measurement curves) of patient 10 in isolation from the air pressure that is used to inflate the inflatable cuff 110. Because the pulse amplitude is detected/measured in isolation of the air pressure that is used to inflate the inflatable cuff 110, the instrument used to measure the pulse amplitude can be specifically designed for detecting a low range of input pressures. In result, the instrument (differential pressure sensor 150) will be more accurate for detecting the low pressures relating to the pulse amplitude in comparison to instruments that are designed to receive a higher range of pressures (e.g., instruments that are designed to be able to receive the pressure used to inflate cuff 110).

In one example technique, BP measurement system 100 can function as follows. The following operative steps can be automatically or semi-automatically controlled by controller 170. For example, in some cases an operator of BP measurement system 100 (e.g., a clinician or patient 10) can simply apply cuff 110 to patient 10 and push a start button of a UI of BP measurement system 100.

Pump 120 inflates cuff 110, and cuff 110 occludes the brachial artery of patient 110 as cuff 110 is inflated. In some cases, cuff 110 is inflated to about 160 mmHg or about 35 mmHg over the actual systolic pressure. Pressure-regulating valve 130 can be used to control the inflation pressure. The closing valve 140 is open during the inflation.

After a full inflation pressure is reached, closing valve 140 is closed. Accordingly, the full inflation pressure is captured in static pressure line 180. Then, differential pressure sensor 150 can be operated to detect the pulse amplitude (BP pulse wave measurement curves) of patient 10 in isolation from the air pressure that is used to inflate the inflatable cuff 110. That is the case because both static pressure line 180 and active pressure line 190 are at the inflation pressure and only active pressure line 190 includes the pulse amplitude (BP pulse wave measurement curves) of patient 10 that result from the occlusion of the brachial artery of patient 10. Therefore, differential pressure sensor 150 will only detect the pulse amplitude (BP pulse wave measurement curves) of patient 10, i.e., in isolation from the air pressure that is used to inflate the inflatable cuff 110. After controller 170 has received the pulse amplitude (BP pulse wave measurement curves) of patient 10 detected by differential pressure sensor 150, closing valve 140 can be opened.

While closing valve 140 remains open, pressure-regulating valve 130 is then used to gradually reduce the inflation pressure of cuff 110. In some cases, the inflation pressure is reduced by about 1-3 mmHG/second in a step-wise manner. When the inflation pressure has reached the desired reduced level, closing valve 140 is closed and the inflation pressure is held steady for a time. Both static pressure line 180 and active pressure line 190 will be pressurized at the reduced level of inflation pressure. During that time, while closing valve 140 remains closed, differential pressure sensor 150 can again be operated to detect the pulse amplitude (BP pulse wave measurement curves) of patient 10 in isolation from the air pressure that is used to inflate the inflatable cuff 110. After controller 170 has received the pulse amplitude (BP pulse wave measurement curves) of patient 10 detected by differential pressure sensor 150, closing valve 140 can be opened again.

While closing valve 140 remains open, pressure-regulating valve 130 is then again used to gradually reduce the inflation pressure of cuff 110 to the next desired level of reduced cuff inflation pressure. Again, when the inflation pressure has reached the desired reduced level, closing valve 140 is closed and the inflation pressure is held steady for a time. During that time, while closing valve 140 remains closed, differential pressure sensor 150 can again be operated to detect the pulse amplitude (BP pulse wave measurement curves) of patient 10 in isolation from the air pressure that is used to inflate the inflatable cuff 110. After controller 170 has received the pulse amplitude (BP pulse wave measurement curves) of patient 10 detected by differential pressure sensor 150, closing valve 140 can be opened again.

The above sequence of operations can be cyclically repeated until the cuff inflation pressure reaches its lowest level for measuring BP. Afterwards, controller 170 can use an algorithm to determine the blood pressure of patient 10 based on the plurality of blood pressure pulse wave measurement curves captured at the various differing levels of cuff inflation pressure.

### EXAMPLES

The novel BP measurement systems described herein were built and tested to prove feasibility and accuracy. Patients on CF LVAD therapy were prospectively studied. The BP was assessed in a stable, supine position shortly after the surgery in the intensive care unit.

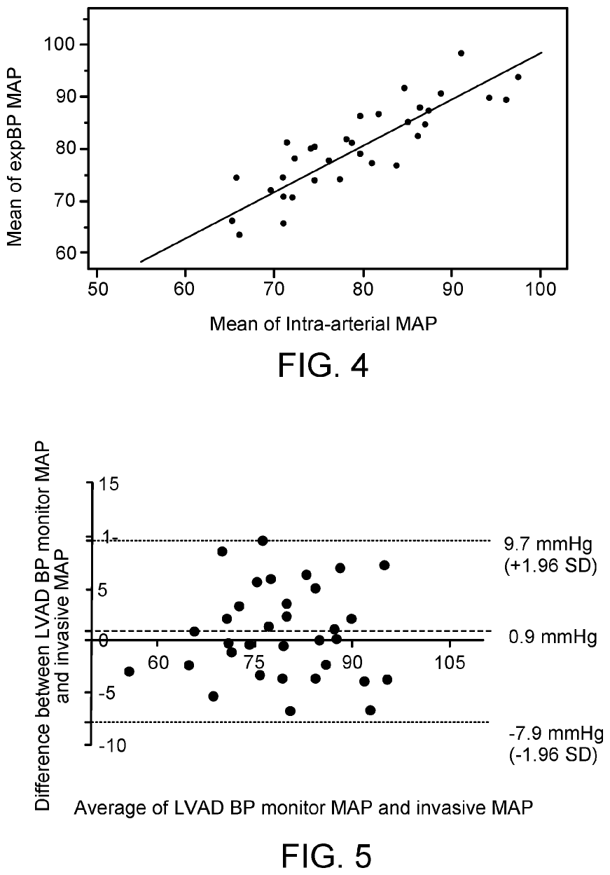

As a baseline for comparison, invasive intra-arterial (I-A) BP was obtained via a radial artery. BP was also assessed with the novel BP measurement systems and techniques described herein. The set-up is depicted in FIG. 3. For further comparison, BP was also assessed using a calibrated sphygmomanometer to perform the Doppler technique. The tests were run within 1 minute intervals of I-A BP.

For the experimental device (i.e., novel BP measurement systems described herein) the arm cuff was inflated on the preset pressure of 165 mmHg for 20 seconds to obtain supra-systolic record of the pulse pressure curve and consequently slowly deflated (2-3 mmHg/sec) to allow assessment of the BP oscillometrically.

For each separate technique, measures were done in triplicate and the average was used for subsequent analyses. Variables were summarized as mean (standard deviation) and frequency (percent) for continuous and categorical measurements, respectively. Bland-Altman (BA) plots were constructed for comparisons of measurement agreement between the novel BP measurement system, Doppler, and I-A BP values, and the Bias+95% confidence intervals were derived. Pearson correlation coefficients were also analyzed. Data was analyzed using JMP Pro 10 statistical software package.

Results

A total of 34 patients (7 females; Age 63±10 years; BMI 27.7±7.4 kg·m²) were tested 3.7±8.4 days post LVAD implantation (18 HM II pumps, 3 HM III and 13 HW pumps). Although BP was successfully assessed in all tested patients by the novel BP measurement systems and techniques described herein (characterized by at least one successful BP reading per subject), the overall success rate reached 94% (6 failed reading from a total of 102 measurements). The Doppler achieved a 100% success rate.

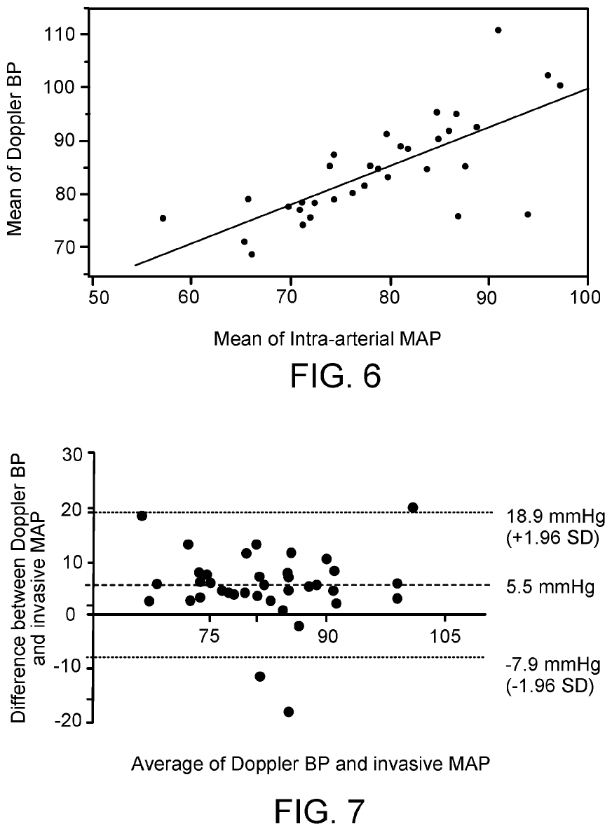

In FIGS. 4 and 5, the mean arterial pressure (MAP) results of the novel BP measurement systems and techniques described herein are compared with the MAP results of the I-A BP measurement method.

In FIGS. 6 and 7, the MAP results of the Doppler technique are compared with the MAP results of the I-A BP measurement method.

To assess validity, mean absolute differences were calculated comparing BP values derived from the novel BP measurement systems and techniques described herein, the intra-arterial line, and Doppler ultrasound in the first cohort; and between the novel BP measurement systems and techniques described herein and Doppler ultrasound measurement in the first and second cohort of subjects. Results, illustrated in Bland-Altman plots show statistically significant difference of MAP means (−5.8±5.7 mmHg) measured by experimental device compared to Doppler method using all captured data and statistically significant difference of MAP means (−6.1±5.7 mmHg) measured by experimental device compared to Doppler method in I-A line cohort of patients. Calculated results also represents statistically significant MAP means (3.7±3.4 mmHg) measured by novel BP measurement systems and techniques described herein to I-A line, and it also shows statistically significant MAP means (9.6±14.4 mmHg) of Doppler method compared to intra-arterial line.

Pulse Pressure Wave Analysis

The data collected using the novel measurement systems and techniques described herein can be analyzed in various manners. In one example, a time domain analysis technique can be used which focuses on pressure pulse wave peak-to-peak data analysis. In another example, a frequency domain analysis technique can be used to perform/obtain spectral analysis of residual arterial pulsatility. Such a frequency domain analysis technique includes a determination of the energy of a group of pulses, or of the signal obtained over a given period of time. Differences between peak-to-peak do not need to be distinguished using the frequency domain analysis technique.

Frequency-decomposition of the non-invasive BP pulse wave offers the ability to determine the presence, and quantify the magnitude, of any residual arterial pulsatility that may be present in LVAD patients post implantation. Specifically, this spectral approach may prove more sensitive in detecting arterial pulsatility when other methods may fail (such as those based in the time domain). In brief, the frequency domain spectral analysis of the non-invasive BP pulse wave can be achieved in accordance with the following example steps:

1. Raw data from the unit is filtered and de-trended.

2. The cardiac frequency (fc) is calculated from automated detection of the peaks in the pulse waveform.

3. The filtered and de-trended pulse waveform is transformed into the frequency-domain, for example via Welch's periodogram (data can be windowed using a Hamming window).

4. The spectral amplitude of the pulse signal is extracted a n multiples of the cardiac frequency.

The primary advantage of this frequency domain spectral analysis approach is that it ensures a high degree of objectivity in determining arterial pulsatility. The user has only to select an appropriate window of time to analyze and report the corresponding spectral amplitude at each multiple (“harmonic”) of the cardiac frequency. This method is straight-forward in its approach, and can be automated and integrated into a wearable and/or portable version of the novel BP measurement systems described herein. This analysis may therefore provide an effective means of monitoring the arterial pulsatility of an LVAD patient across the course of their clinical management, yielding opportunities for enhancing the clinical management and optimization of care in this patient population.

Conclusions

Using the invasively obtained I-A BP as the baseline, the results demonstrate closer agreement (more accuracy) of mean arterial BP assessed using the novel BP measurement systems described herein than using the Doppler technique. FIG. 8 shows the correlation between a particular blood pressure pulse wave measurement curve measured by: (i) the invasively obtained I-A BP (curve 200) and (ii) the novel BP measurement systems described herein (curve 210).

With a satisfactory “success rate” of measurement attempts in challenging hemodynamic conditions of LVAD patients shortly after the surgery, the novel BP measurement systems described herein also provides systolic and diastolic BP compared to a single BP value delivered by the Doppler technique. Translation of the novel BP measurement systems described herein into clinical practice could potentially lead to simplification of BP monitoring, allowing for improved BP control in the LVAD population, and in turn this could potentially positively impact adverse events rates associated with a poor BP control. Easy to operate and accurate non-invasive BP measurement assessment might contribute to the better outpatient care, decreasing rate of complications related to BP control.

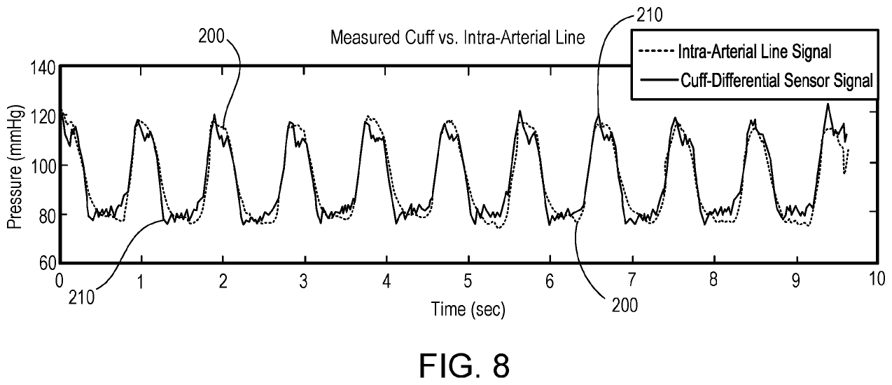

FIGS. 9-12 illustrate the correlation between BP measurement results of: (i) invasively obtained I-A BP and (ii) non-invasively obtained BP (using the novel BP measurement systems and the aforementioned frequency domain spectral analysis approach).

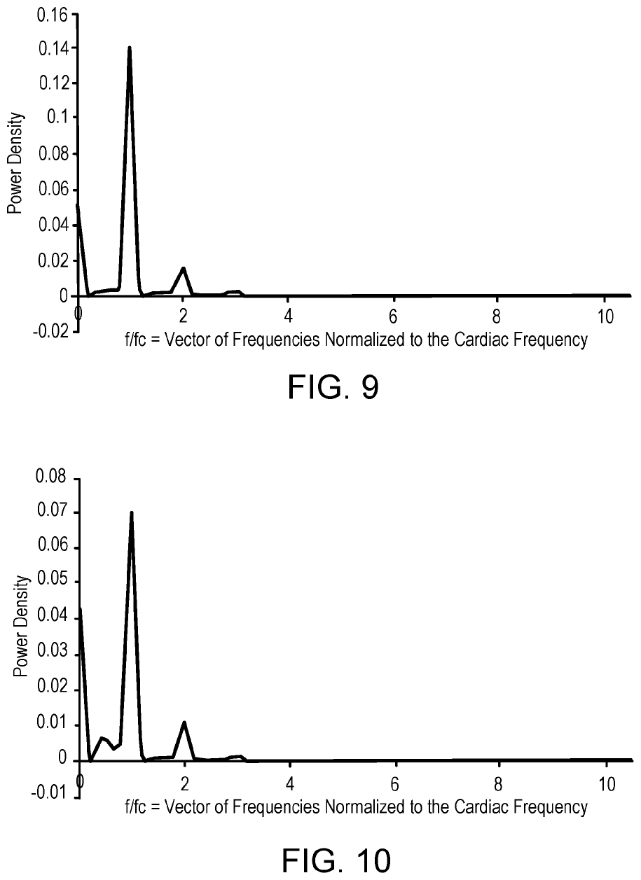

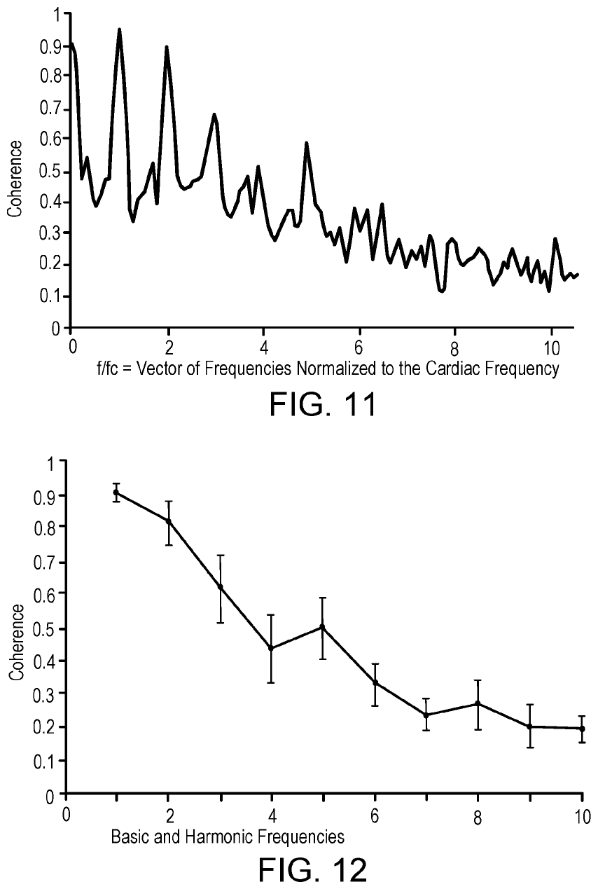

FIG. 9 illustrates the frequency characteristics of a blood pressure signal obtained invasively using an intra-arterial (I-A) sensor and characterized via a normalized power spectral density. FIG. 10 illustrates the frequency characteristics of a blood pressure signal obtained non-invasively using novel measurement systems and techniques described herein (e.g., system 100) characterized via a normalized power spectral density. FIGS. 11 and 12 illustrate the correlation between the results of FIGS. 9 and 10. Strong correlation between the frequencies of the two techniques is illustrated (especially at the first (about 95%), second (about 90%), and third (70%) harmonic frequencies). This provides a high level of confidence that the non-invasively obtained BP using the novel BP measurement systems and the aforementioned frequency domain spectral analysis approach provides accurate BP measurement results.

Discussion

Compared to the oscillometric BP monitors used for the general non-LVAD population, measurement of absolute BP values is realized during gradually deflating cuff pressure using a lower speed, circa 2 mm Hg/sec from the initial inflation to suprasystolic pressure (circa 160 mm Hg). Additionally, a differential pressure sensor is utilized to more accurately record weak oscillometric pulsations of CF LVAD patients. One input of the sensor is supplied with a pressure signal without oscillometric pulsations (“static component”) whose value corresponds with a cuff pressure see arm (A) in FIG. 1. Second input is supplied with cuff pressure including superimposed oscillometric pulsations, see arm (B) in FIG. 1. The inputs of the sensor are controllably separated by closing valve (CV) during step deflation. Static (cuff) pressure is regulated via a servo-regulatory valve (RV) that is controlled by microprocessor. At the output of the sensor are thus, during the step defaltion, only oscillometric pulsations at different pressure levels (steps) and oscillometric pulsation envelope is then compiled. Measured values were continuously stored in the memory of the device at a sampling rate Fs=400 Hz. No other filtering was used during hardware measurement phase. This method allows up to 20 times more sensitive recording of oscillometric pulsations compared with standard oscillometrical monitors.

A single inflatable arm cuff with size based on the arm circumference with 5 cm of overlap (3 cm above cubital fossa) was wrapped around the left arm over brachial artery and connected to the prototype of LVAD BP monitor (the novel BP measurement system described herein).

Compared to the oscillometric BP monitors used for the general non-LVAD population, measurement of absolute BP values is realized during gradually deflating cuff pressure using a lower speed, circa 2 mm Hg/sec from the initial inflation to suprasystolic pressure (circa 160 mm Hg). Additionally, the novel BP measurement system described herein includes a differential pressure sensor that is utilized to more accurately record weak oscillometric pulsations of CF LVAD patients. One input of the differential pressure sensor is supplied with a pressure signal without oscillometric pulsations (“static component”) whose pressure value corresponds with the cuff pressure. A second input is supplied with cuff pressure including superimposed oscillometric pulsations from the patient. At the output of the differential pressure sensor are thus only oscillometric pulsations.

For the precise assessment of the blood pressure pulsatility each measurement with the experimental cuff method consisted of the following steps: the cuff was pressurized to 30 mmHg above previously assessed systolic BP for 30 seconds. In stop-flow conditions, continuous pressure signal generated by the artery and transmitted through the cuff (used as a primary pressure sensor) was recorded by the prototype of LVAD BP monitor (the novel BP measurement system described herein) and the raw BP curve data were recorded on a prototype BP monitor. Measured values were continuously stored in the memory of the device at a sampling rate Fs=400 Hz. No other filtering was used during hardware measurement phase. Raw signal was analyzed using two separate features and validated with the simultaneously recorded arterial BP signal using Power Lab (AD Instruments).

Frequency-decomposition of the continuous non-invasive BP signal offers the ability to determine the presence, and quantify the magnitude, of any residual arterial pulsatility that may be present in LVAD patients post implantation. Specifically, this spectral approach may prove to be more sensitive in detecting arterial pulsatility in patients a with very low pulse pressure amplitude when other methods may fail (such as those based in the time-domain). For validation purposes, signals from the novel BP measurement system described herein and from the I-A BP signal were processed identically from corresponding time intervals (about 20 seconds). In 10 subjects, preliminary data analysis revealed a strong relationship between the power spectral density of intra-arterial and the cuff pressure signals at the 1^{st} and 2^{nd} harmonics of the cardiac frequency (i.e., heart rate).

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A system for measuring a blood pressure of a patient, the system comprising:
an inflatable cuff configured to releasably surround an arm of the patient and to occlude a brachial artery of the patient while the inflatable cuff is inflated;
a pressure-regulating valve in fluid communication with the inflatable cuff;
a second valve in fluid communication with the inflatable cuff and with the pressure-regulating valve; and
a differential pressure sensor arranged to detect a blood pressure pulse wave measurement curve of the patient.

2. A system for measuring a blood pressure of a patient, the system comprising:
an inflatable cuff configured to releasably surround an arm of the patient and to occlude a brachial artery of the patient while the inflatable cuff is inflated;
a pressure-regulating valve through which air passes to inflate and deflate the inflatable cuff;
a second valve in fluid communication with the inflatable cuff and with the pressure-regulating valve; and
a differential pressure sensor arranged to detect a differential pressure of fluid lines on opposing sides of the second valve.

3. The system of claim 1, further comprising:
a pump to supply air to inflate the inflatable cuff, wherein the pressure-regulating valve is located along an air supply line between the pump and the inflatable cuff.

4. The system of claim 1, further comprising:
a controller configured to receive signals from the differential pressure sensor and to determine the blood pressure based on the signals.

5. The system of claim 4, wherein the controller controls opening and closing of the second valve.

6. The system of claim 4, wherein the controller controls the pressure-regulating valve to control air pressure in the inflatable cuff.

7. The system of claim 4, wherein the controller controls a pump that supplies air to inflate the inflatable cuff.

8. A method of measuring blood pressure of a patient, the method comprising:
inflating an inflatable cuff of a blood pressure measurement system to occlude a brachial artery of the patient, wherein the blood pressure measurement system further comprises:
a pressure-regulating valve in fluid communication with the inflatable cuff;
a second valve in fluid communication with the inflatable cuff and with the pressure-regulating valve; and
a differential pressure sensor arranged to detect a blood pressure pulse wave measurement curve of the patient;
detecting, by the differential pressure sensor and while the brachial artery is at least partially occluded, a plurality of blood pressure pulse wave measurement curves; and
determining the blood pressure based on the plurality of blood pressure pulse wave measurement curves.

9. The method of claim 8, wherein the blood pressure measurement system further comprises a controller that receives signals from the differential pressure sensor corresponding to the plurality of blood pressure pulse wave measurement curves, and wherein said determining the blood pressure is performed by the controller.

10. The method of claim 9, wherein the controller controls the pressure-regulating valve to vary air pressure in the inflatable cuff.

11. The method of claim 9, wherein the controller controls opening and closing of the second valve.

12. The method of claim 8, wherein said detecting the plurality of blood pressure pulse wave measurement curves is performed while the second valve is closed and such that the differential pressure sensor measures a difference in pressures at opposing sides of the second valve.

13. The method of claim 8, wherein said detecting the plurality of blood pressure pulse wave measurement curves is performed without pressure effects of air pressures that inflate the inflatable cuff.

14. The system of claim 2, further comprising:
a pump to supply air to inflate the inflatable cuff, wherein the pressure-regulating valve is located along an air supply line between the pump and the inflatable cuff.

15. The system of claim 2, further comprising:
a controller configured to receive signals from the differential pressure sensor and to determine the blood pressure based on the signals.

16. The system of claim 15, wherein the controller controls opening and closing of the second valve.

17. The system of claim 15, wherein the controller controls the pressure-regulating valve to control air pressure in the inflatable cuff.

18. The system of claim 15, wherein the controller controls a pump that supplies air to inflate the inflatable cuff.
